(19) United States
(12) Patent Application Publication
(10) Pub. No.: US 2006/0155170 A1
(43) Pub. Date: Jul. 13, 2006

(54) Guided retractor and methods of use

(75) Inventors: Shaun Hanson, West Chester, PA (US); Jeffrey Larson, Coeur d'Alene, ID (US); Theodore Bertele, Longmont, CO (US); Louis Greenberg, Boulder, CO (US); Scott Schorer, Niwot, CO (US)

Correspondence Address: JONES DAY, 222 EAST 41ST STREET, NEW YORK, NY, 10017-6702, US

(73) Assignee: Synthes Spine Company, LP

(21) Appl. No.: 11/260,542

(22) Filed: Oct. 26, 2005

Related U.S. Application Data
(63) Continuation-in-part of application No. 10/645,136, filed on Aug. 20, 2003.
(60) Provisional application No. 60/433,343, filed on Dec. 13, 2002.

(51) Int. Cl. A61B 1/32

(52) U.S. Cl. 600/201

(57) ABSTRACT

The present invention provides methods and apparatus in which a surgical retractor comprises a plurality of mechanically coupled tissue retaining walls that are guided into position along one or more guides previously implanted into the patient. The walls are preferably coupled by pivots, so that separating some of the walls from one another opens an operating space. There are preferably two guides, which are driven or screwed into the pedicles of vertebrae, or other bone. Since practical considerations will usually mean that the guides are completely parallel to one another, the retractor has oversized channels to receive the guides. The channels are best disposed in a frame, which also serves to hold lock the walls apart. Various convenience features are contemplated including a web disposed between the walls, which expands as the walls are separated. Also contemplated are projections from near the bottoms of one or more of the walls, which can alternatively or additionally help to hold the underlying tissue in place.

### CROSS-REFERENCE TO RELATED APPLICATIONS

This application is a continuation-in-part of U.S. patent application Ser. No. 10/645,136, filed on Aug. 20, 2003, which claims priority to U.S. Provisional Application Ser. No. 60/433,343, filed on Dec. 13, 2002, the contents of each expressly incorporated by reference herein.

### FIELD OF THE INVENTION

The field of the invention is surgical retractors.

### BACKGROUND OF THE INVENTION

Many types of surgical retractors are known. The simplest devices are tubular probes, or probes adapted with a paddle or other somewhat flatter surface. Recent embodiments of that concept are depicted in U.S. Pat. No. 6,206,826 to Mathews et al. (March 2001). More complicated retractors utilize scissors, bow string, or screw-jack expanders that operate against mating paddles. Those retractors have the advantage of being able to lock the paddles in place, leaving at least one of the surgeon's hands free for other actions. See e.g., U.S. Pat. No. 6,471,644 to Sidor (October 2002). Still other retractors are self opening, including Cosgrove et al., U.S. Pat. No. 6,162,172 (December 2000). All cited patents herein are incorporated herein by reference.

While undoubtedly useful in many respects, none of the above-mentioned retractors are readily fixed in position relative to one or more bones. U.S. Pat. No. 5,027,793 to Engelhardt et al. (July 1991) addresses that need to some extent, by providing spikes on the bottom of a retractor wall, and further providing spikes that can be driven into the bone. The contemplated use is to resect the operating area down to the bone, position the retractor, and then pound both the retractor and the spikes into place.

A problem remains, however, in that the resection required to properly position the retractor can cause considerable trauma to the overlying and surrounding tissues. Another problem is that multiple retractors are needed to retain tissue pushing into the operating area from different directions. The Engelhardt et al. retractor, for example, did not have to address that issue because the preferred application was acetabular surgery, in which the major encroachment was from gluteus muscles that are all substantially superior to the operating site.

In spinal and some other surgeries these problems can be especially severe. Thus, there is still a need to provide methods and apparatus in which an operating space can be positioned and opened with respect to specific areas of bone, while reducing trauma to surrounding tissue.

### SUMMARY OF THE INVENTION

To that end the present invention provides methods and apparatus in which a surgical retractor comprises a plurality of mechanically coupled tissue retaining walls, which are guided into position along one or more guides previously implanted into the patient.

Preferred embodiments utilize two main walls, and four smaller walls, one on each of the ends of the two main walls. In such embodiments all of the walls are coupled by pivots, such that the faces of the two main walls can be moved towards or apart from each other to open or close an operating space. The faces of at least the main walls are preferably flat, but can be any other suitable shape, including convex. The invention is particularly suited for operating on or near curved bony surfaces, and the bottoms of the walls can be compliant (i.e., advantageously adapted to fit and/or conform to the bone surface below).

There are preferably two guides, which are driven or screwed into the pedicles of vertebrae, or other bone. The various guides can be implanted into different bones, or different areas of the same bone. Since practical considerations will usually mean that the guides are parallel to one another, the retractor has oversized channels to receive the guides. The channels can be circular in cross section, but are more preferably elongated into an oblong or other slotted shape.

The channels are best disposed in a frame, which also serves to hold lock the walls apart. Any suitable devices can be used to move apart the main walls to open the operating space, including for example a simple wedge or T-bar, or a mechanism disposed on the frame. The frame can be held in place relative to the guides by wires, nuts, clamps, and so forth.

Various convenience features are contemplated including a web disposed between the walls, which expands as the walls are separated. The web can be cut, torn, bent away, or otherwise manipulated to expose the tissue below. Also contemplated are projections from near the bottoms of one or more of the walls, which can alternatively or additionally help to hold the underlying tissue in place, and can similarly be removed in any suitable manner from the corresponding wall. The frame or other portion of the retractor can be transparent to aid in surgeon visualization.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is a perspective view of a retractor according to the inventive subject matter, in an open configuration;

FIG. 2 is a perspective view of the retractor of FIG. 1, disposed in a closed configuration;

FIG. 3 is a perspective view of the back and spine of a patient, in which finger dissection is being employed to locate a pedicle of a vertebra;

FIG. 4 is a horizontal cross-sectional view of a vertebra, showing use of an awl to punch a guide hole into a pedicle;

FIG. 5 is a horizontal cross-sectional view of the vertebra of FIG. 4, in which a screw is being screwed into the hole created in FIG. 4;

FIG. 6 is a perspective view of the back and spine of a patient in which the closed retractor of FIG. 2 is being fitted onto the guides implanted into adjacent vertebrae;

FIG. 7 is a perspective view of the back and spine of the patient of FIG. 6 in which the retractor is being opened by an opening tool;

FIG. 8 is a perspective view of the back and spine of the patient of FIG. 6 in which the retractor has been opened, and the web is being removed to expose various fingers and the underlying tissue;

FIG. 9 is a perspective view of the back and spine of the patient of FIG. 6 in which the retractor has been opened, and various fingers (bottom tissue retainers) are being removed;

FIGS. 10A-10B are perspective views of another embodiment of a retractor in an open position and attached to a spine;

FIG. 11A is a top view of a retractor in a closed position that has been lowered over guides;

FIG. 11B is a top view of the retractors of FIGS. 10A-10B;

FIG. 12A is a top view of the retractor used in FIGS. 10A-11B in an open position;

FIG. 12B is a front view of the retractor of FIG. 12A;

FIG. 13A is a side view of an embodiment of a guide for use with the retractor of FIGS. 10A-12B;

FIG. 13B is a cross-sectional view of the guide of FIG. 13A;

FIG. 14A is a side view of another embodiment of a guide for use with the retractor of FIGS. 10A-12B;

FIG. 14B is a cross-sectional view of the guide of FIG. 14A;

FIG. 15 is perspective view of an embodiment of a connector for use with the retractor of FIGS. 10A-12B;

FIG. 16 is a perspective view of an embodiment of a spreader for use with a retractor; and

FIG. 17 is a perspective view of another embodiment of a retractor frame.

### DETAILED DESCRIPTION OF THE INVENTION

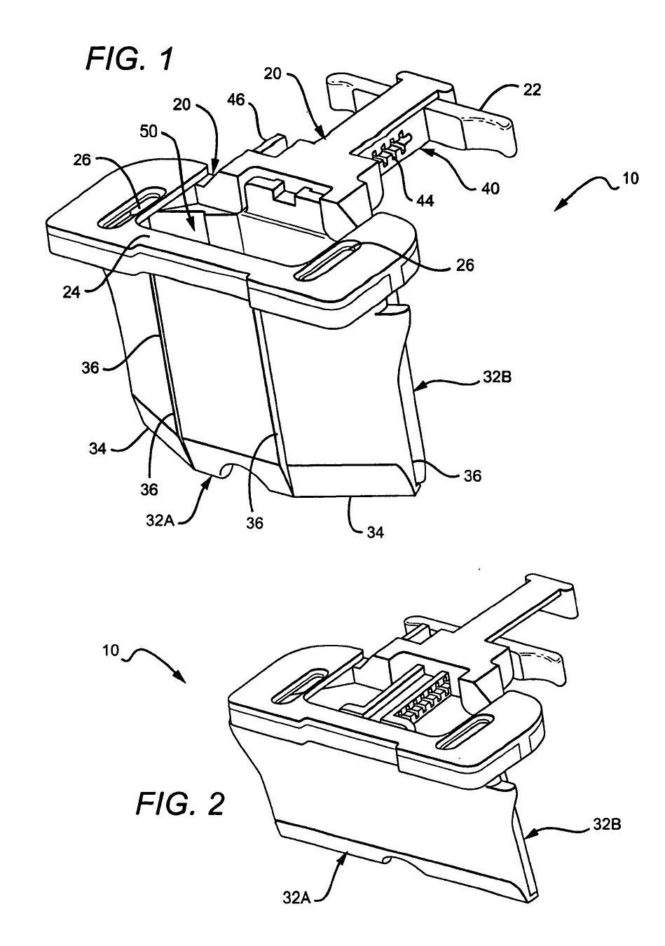

FIG. 1 generally depicts a retractor 10, having a frame 20, major walls 32A, 32B and minor walls 34, and a locking/opening mechanism 40. The various walls 32A, 32B, 34 are coupled by six hinges 36, and in the open position depicted in the figure cooperate to define an operating space 50.

The frame 20 can be any suitable size and shape according to the particular applications, with larger frames being generally more useful for larger incisions. For posterior lumber surgery on adult humans, the overall dimensions of an especially preferred frame are about 5.5 cm in depth, 3.5 cm in length, 3 cm in width. Frame 20 is preferably made from Delrin™, but can be made of any suitable material, especially a nontoxic polymer such as polyethylene. The frame 20 can advantageously be colored to reduce glare from operating room lighting, and some or all of the frame can be relatively transparent.

Frame 20 generally comprises a handle portion 22 that includes the locking mechanism 40, and a perimeter 24 around the operating space 50. The locking mechanism 40 is shown as a ratchet, but all other suitable locking mechanisms are also contemplated, especially those that provide for a high degree of reliability and ease of operation. At least one of the walls 32A, 32B, 34 is preferably coupled to the perimeter 24 using a pin (not shown).

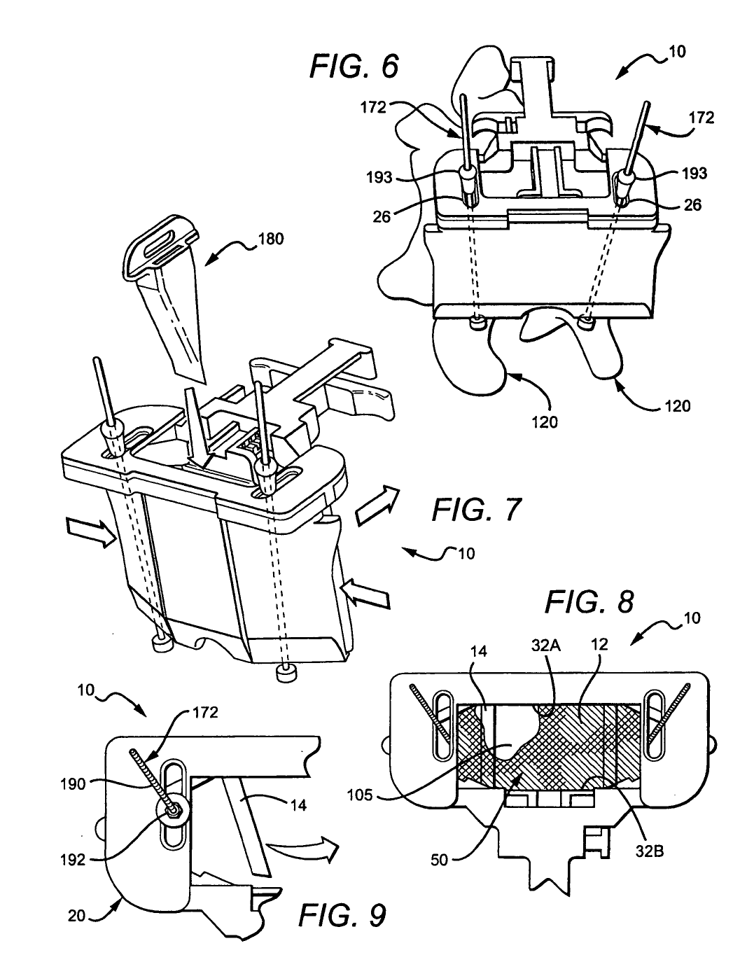

Channels 26 are located on opposite sides of the perimeter 24, and are each sized to receive one of the guides 172 (see FIGS. 4-9). The system is designed to work with a wide range of pedicle screw or other bone fixation systems, and with various numbers of guides, regardless of the specific relationship between screw and guide. In addition, the passageways defined by the channels 26 should be oversized with respect to the outside diameters of the shafts of the guides 172 so that the channels 26 can receive guides 172 that are out of parallel or in some other manner not perfectly aligned with each other. In preferred embodiments the channels define a passageway having a diameter of about 5 to 15 mm, whereas the guides 172 (see FIGS. 5, 6) preferably have a corresponding diameter of about 4 to 6 mm. All ranges set forth herein should be interpreted as inclusive of the endpoints.

As with other components, the various walls 32A, 32B, 34 are preferably made of a biocompatible material, and here again they can have any suitable sizes and shapes, depending on the surgical site or sites for which they are intended. The currently preferred material for retractor walls is polypropylene. Walls 32A, 32B, 34, for example, can be mostly rectangular in vertical cross-section as shown, with bottoms of at least the major walls 32A, 32B curved to accommodate specific bone shapes, such as that of the spinous processes of the vertebrae in spinal surgery. It is also contemplated that the bottoms of at least the major walls 32A, 32B can be pliable, to conform at least partially to projections and depressions of the underling bone. Walls 32A, 32B, 34 are depicted in the figures as having flat sides, but alternatives may be bowed outwardly (convex), inwardly (concave), or may have any other suitable horizontal cross-section.

One or more of the walls (not shown) can even be inflatable, made out of balloons that define the opening. Of course, the walls 32A, 32B, 34 must be sturdy enough, and therefore thick enough, to withstand the expected forces placed upon them. On the other hand the walls 32A, 32B, 34 are preferably not so thin that they would cut into the tissue below during deployment. On the other hand, the walls 32A, 32B, 34 should not be so thick as to significantly interfere with the size of the operating area. Preferred thickness is from about 3.5 mm to about 5 mm at the thickest point, tapering down to a thickness of 1.5 mm-3 mm at the bottom of each wall. The walls can also be nested in any suitable manner, which simply means that a portion of one wall extends around a portion of another wall.

The hinges 36 are shown as continuations of the walls 32A, 32B, 34. Indeed all of the walls and hinges can be molded as a single piece, with each of the hinges 36 being formed as an especially thin edge of a wall. This is effectively a “living hinge” that can handle multiple openings, using material properties of polypropylene. All other suitable configurations of hinges are also contemplated. For example, instead of four minor walls 34, the major walls 32A, 32B could be coupled by only a single outwardly bowed, flexible piece (not shown) at each end. Certainly the total number of walls can be greater or less than 6.

The term “wall” is used herein in a very broad sense, to mean any sort of tissue retaining barrier, generally longer than tall, and considerably taller than thick. Retractor 10 could thus be termed a “linear retractor” to distinguish it from point retractors that are basically pen-shaped probes. But neither the retractor as a whole nor any of the walls are necessarily linear. The term certainly does not require that the wall be so thin as to constitute a cutting blade. Nor does the term “wall” require that the sides thereof be completely patent. The sides of the walls may be pitted or indented as would occur if the sides had a mesh coating (not shown), and the sides may even have through holes (not shown).

Locking/opening mechanism 40 is shown as a typical ratcheting type mechanism, with teeth 44, and having a release 46. Frame 20 can have both a locking mechanism and an opening mechanism (not shown), or either one by itself. There are numerous other locking and/or opening mechanisms known to the field, and presumably others will become known in the future. It is contemplated that any suitable locking and/or opening mechanisms can be used.

Operating space 50 will be larger or smaller depending on the sizes and shapes of the walls, and the extent to which the walls are separated out from one another. Preferred area of the operating space 50 is between 7 cm² and 14 cm².

FIG. 2 generally depicts the retractor 10 of claim 1, disposed in a closed configuration. The terms “closed” and “open” with respect to configurations of the retractor 10 are relative. Thus, closed merely means substantially closed, but does not require complete closure, so that the walls 32A, 32B are juxtaposed. In a closed position the walls 32A, 32B may well be separated by up to 1 mm or more. Similarly, in a contemplated open configuration, walls 32A, 32B would likely be separated by at least 1.5 cm, but may be separated by up to 2.3 cm or more.

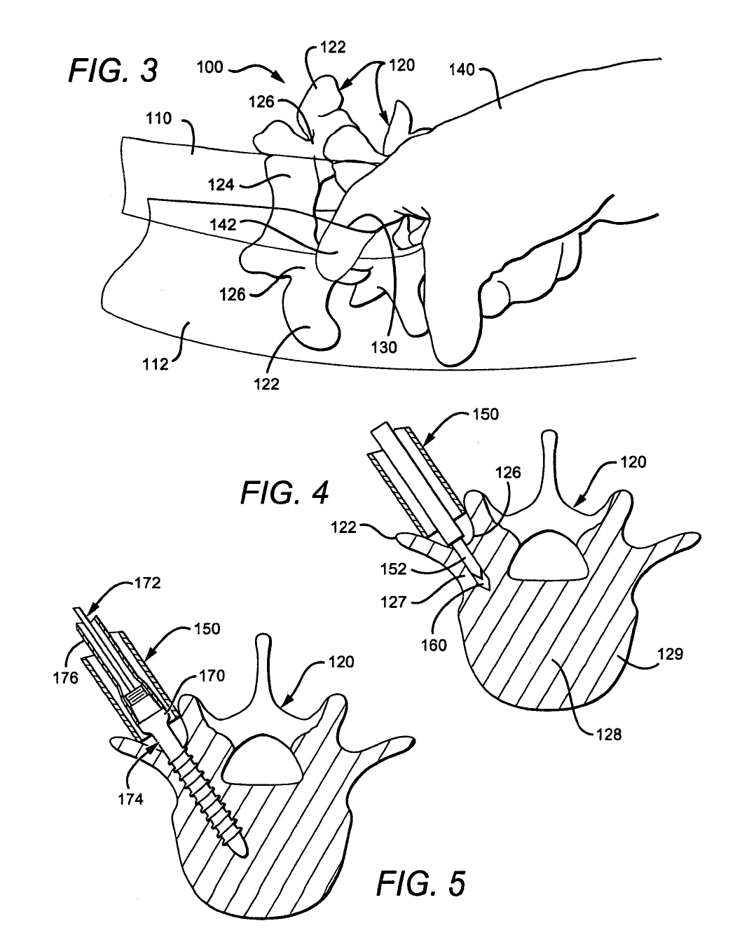

FIG. 3 generally depicts a portion of the spine 100 of a patient, in which the paraspinous muscles are designated schematically by semitransparent bands 110, 112, respectively. The spine 100 includes vertebrae 120, each of which includes transverse processes 122, spinous processes 124, and pedicles 126. An incision 130 has been made, and a finger 142 of hand 140 is being used to dissect through the muscle and locate one of the pedicles 126. Of course a wedge, probe or other tool could be use in place of or in addition to the finger 142 to locate the pedicles.

FIG. 4 generally depicts cannula 150 that positions an awl or probe 152 for use in producing a hole 160 in the pedicle 126. The awl 152 can be manually pushed or otherwise forced through the cortex 127 of the pedicle. Cannula 150 is preferably made of radiolucent material such as plastic or carbon fiber, while awl and probe 152, and other tool attachments and inserts are all preferably made of metal such as surgical steel, titanium, or other durable, radio opaque material. Positioning the cannula 150 can be aided by fluoroscopy or other visualization technique.

In preferred methods, the awl 152 is withdrawn, and a longer, thinner probe (not shown) is inserted through the pedicle 126 into the softer medulla 128 of the body 129 of the vertebra 120. The longer pin is then withdrawn, and in FIG. 5 a screwdriver 176 is used to insert a screw 174. The screw has a head 170, which holds a guide 172 in place. The screwdriver 176 is then removed, leaving the screw 174 implanted into the vertebra 120, and guide 172 rotatably attached to the top of screw 174. The process is repeated to insert another guide 172 into another area of bone, which in the case of spinal surgery is most likely the pedicle of an immediately superior or inferior vertebra on the same side. In other surgeries (not shown), the second, or possibly even a further guide, can be inserted into a different location of the same bone as received the first guide.

In FIG. 6 the guides 172 that are implanted into adjacent vertebrae 120 have been inserted into the channels 26 of the closed retractor 10. Those skilled in the art will realize that the channels can have other configurations besides those shown in the drawing, and can be multi-level rather than simply a single level.

In FIG. 7 the retractor 10 is being opened by an expander 180, which is manually inserted between the opposing walls to produce and widen a gap between them. In this figure the expander generally comprises a wedge with a handle. The expander 180 is preferable over using unassisted fingers because it involves a mechanical advantage. Alternatively, the retractor can be opened using a thumb and fingers-opposing force method using the handle 22 and frame 20. There are numerous alternatives which may or may not involve any mechanical advantage, including for example a T handle coupled to a shaft and a cam (not shown).

In FIG. 8 the retractor 10 has been opened to reveal a web 12 positioned between walls 32A, 32B and 34. The web 12 is preferably a thin, flexible sheet of latex or other biocompatible plastic, which can be easily cut, ripped, or in some other manner disrupted to expose various retaining fingers 14 and the underlying tissue 105. Web 12 is shown as covering the entire floor of the operating space 50, but it could alternatively cover a lesser space, and could extend between or among different walls. The fingers 14 are depicted as extending from or rotating out below the web 12, but some or all of the fingers 14 could alternatively be positioned above the web 12. Each of web 12 and fingers 14 are certainly optional.

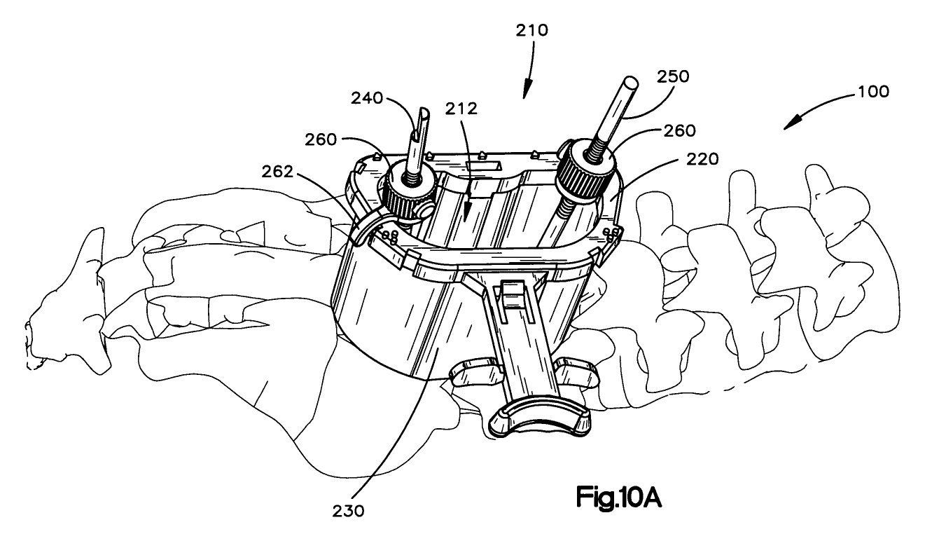

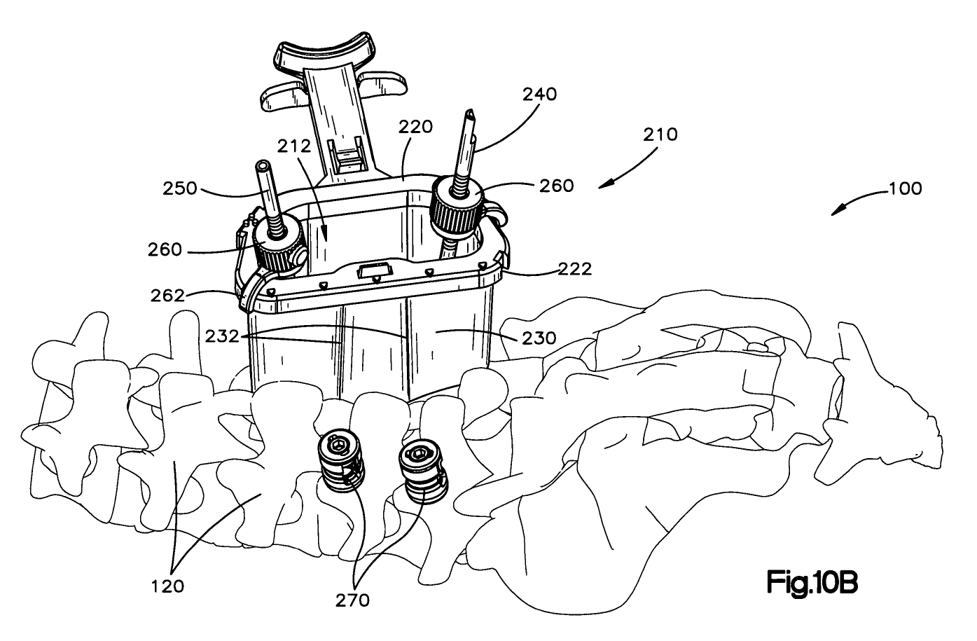

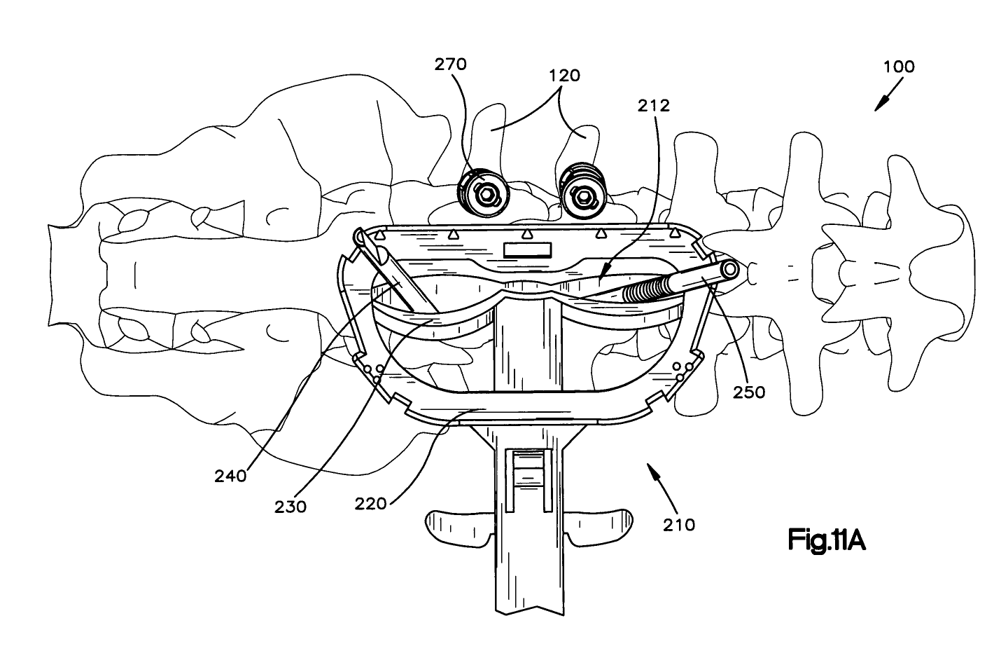

In FIG. 9 the retractor 10 has been opened, and various fingers 14 are being removed. Such removal can be accomplished in any suitable manner, including by cutting (as with a scalpel or scissors), bending by hand or with a tool, and so forth. There may be wide fingers, narrow fingers, long or short fingers, closely spaced or widely spaced fingers, flat or rounded fingers, and so on (not shown). Where fingers are used, they are preferably molded as continuous extensions of the walls.

Also shown in FIGS. 8 and 9 are threads 190 the guides 172 can be at least partially threaded, and can thereby that receive wing nuts or other correspondingly threaded pieces 192 that assist in anchoring the frame 20 to the guides 172. In alternative configurations one could use non-threaded lock down pieces such as finger clamps 193. In especially preferred embodiments alternative templates (not shown) can be placed on top of the frame, and held in place using the wing nuts, finger clamps, or other hold-down devices. The frame can also be used to hold additional devices, such as suction or lighting, introduced into the field 50 and held in place by a coupling device on the frame 20.

Preferred methods of inserting a tissue retractor 10 into a patient involve the steps of providing a retractor 10 having paired tissue retracting surfaces (such as on walls 32A, 32B, 34) and first and second guide receiving areas (such as channels 26); percutaneously or otherwise implanting first and second guides (such as guides 172) into different areas of bone in the patient; then positioning upper ends of the first and second guides through the first and second guide receiving areas, respectively, then fully inserting the retractor down the guides and into the patient, effectively splitting the muscle; and finally moving the tissue retracting surfaces apart from one another to open the operating space. These methods are especially useful where one or more of the guides are screws, which are implanted into very specific anatomical structures such as the pedicles of vertebrae. The contemplated methods are also extremely useful in opening operating spaces overlying adjacent bones. Especially preferred methods optionally employ nuts, clamps, or other readily attachable and tightenable mechanisms to stabilize the retractor 10 on the guides.

From the description above, it should now be apparent that the novel methods and apparatus disclosed herein turn the normal retracting procedure on its head. Instead of positioning the retaining wall or walls and then holding them in place by implanting spikes or posts into the bone, the present procedure implants the spikes or posts, and then uses them as guides to position the retaining wall(s).

The advantages of turning the procedure around are significant. Among other things, this new procedure allows the surgeon to exactly position the retractor 10 at the intended operative site because the positioning can be done precisely with respect to underlying bony structures (e.g., the pedicle 126 of a vertebra). The screws are implanted where the surgeon wants them, and the guides 172, being attached to the top of the screws guide the retractor down into the desired anatomy, splitting the muscles, and defining a operating site 50 within the walls 32A, 32B and 34. After that the operating site 50 is opened, giving the surgeon the desired exposure needed to conduct the surgery., without excess retraction and resulting tissue destruction.

Another advantage is that these new methods and apparatus speed up the procedure and makes more efficient use of resources relative to the prior art. Among other things, after the guides 172 and screws 174 are placed and the retractor 10 is attached and opened, there is no more need for fluoroscopy, which can be moved along to a different room.

Still other advantages involve convenience and reduction in surgeon stress. The novel methods and apparatus make it mentally easier on the surgeon. After the screws 174 are in, in the first part of the procedure, everything else in terms of opening the operating site is fairly straightforward. This helps the surgeon relax mentally and physically.

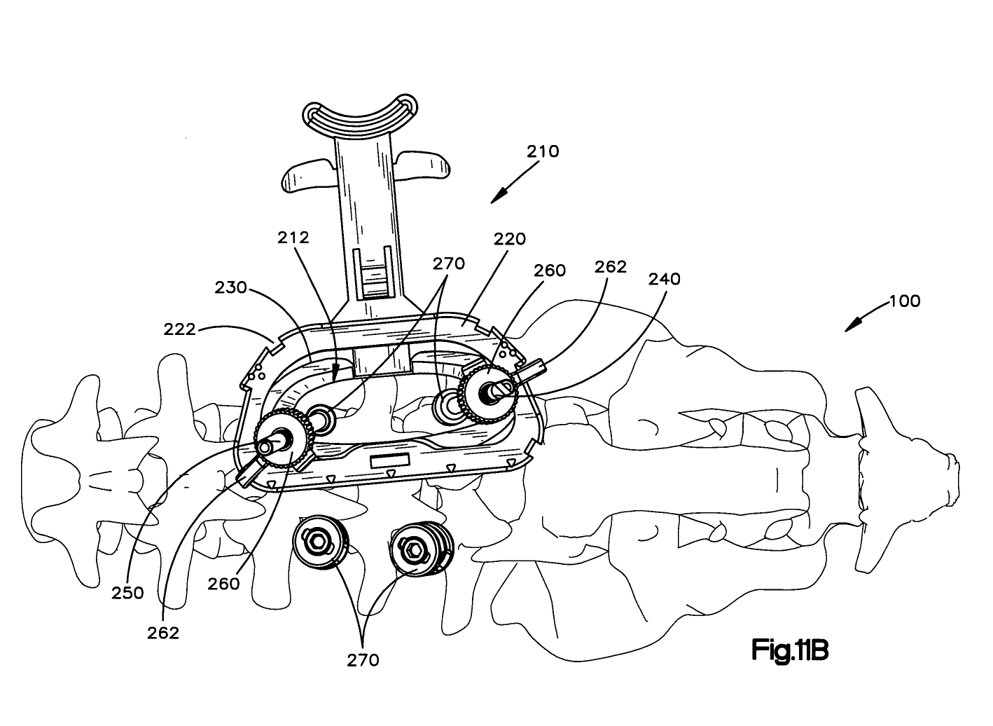

Another embodiment of a retractor 210 is shown in FIGS. 10A-12B. As seen in the perspective views of FIGS. 10A-10B, retractor 210 may be comprised of a frame 220 and a blade portion 230, which may form an opening 212 used to perform surgical procedures therein. Blade portion 230 may also have an insertion portion configured to be at least partially inserted into a patient. Retractor 210 may be associated with a portion of a spine 100 via guides 240, 250 and connectors 260. Connectors 260 may have an attachment portion 262 for selective attachment to the frame 220 via indentations 222. Blade portion 230 may have at least one blade, and a plurality of blades may be separated by a hinge 232. Guides 240, 250 may be (as in the embodiments shown in FIGS. 10A-11B) two different types of guides, or they may be the same types of guides. Guides 240, 250 may be associated with bones screws 270, which may be inserted into the pedicle of a vertebrae 120, and which may or may not have an attachment portion connected to the heads of such screws 270.

FIG. 11A shows a top view of a retractor 210 slidably engaged with guides 240, 250, before a connector 260 has been utilized. In this embodiment, blade portion 230 is in a closed configuration, and retractor 210 has been lowered toward the spine 100 over guides 240, 250. Guides 240, 250 may be within the opening 212 created by the blades of blade portion 230. Before connectors 260 are attached to guides 240, 250 and the frame 220, retractor 210 may be slidably engaged with guides 240, 250, such that the distance between retractor 210 and spine 100 may be altered to a desired distance.

FIG. 11B shows a top view of a retractor 210 in an open position, and fixedly associated with guides 240, 250 via connectors 260. As can be seen in this embodiment, connectors 260 have been used to associate guides 240, 250 with frame 220, and the blades of blade portion 230 have been opened to create a larger opening 212 than in FIG. 11A. Guides 240, 250 are seen in this embodiment to engage (either directly or indirectly) bone screws 270. Preferably, guides 240, 250 can assume a variety of angulations in relation to a bone screw 270. This may be achieved by providing a polyaxial association between guides 240, 250 and a bone screw 270. Bone screws 270 may be introduced into the body and into engagement with the body by numerous suitable methods, including utilizing a cannula and/or dilator to access the desired insertion location by creating an access port.

In use, and in reference to FIGS. 11A-11B, guides 240, 250 are attached to bone screws 270, such that guide 240, 250 project away from the spine 100. While two guides 240, 250 are shown, it is contemplated, that one, three, or more guides 240, 250 may be utilized. Moreover, although two bone screws 270 are shown for use with retractor 210, it is expressly contemplated that one, three, or more bone screws 270 may be used. The number of guides 240, 250 may be less, the same, or more than the number of bone screws 270. As stated above, guides 240, 250 preferably are polyaxially and/or pivotably associated with a bone screw 270.

After guides 240, 250 are attached to bone screws 270, retractor 210 may be lowered over the guides 240, 250 such that the guides 240, 250 pass through the opening 212 of blade portion 230, and project upwardly away from the retractor 210. Preferably, blade portion 230 is in a closed position when the retractor 210 is lowered over guides 240, 250. As stated above, it may be preferable to do so to create a generally linear leading edge of the blade portion 230 to increase ease of insertion into an incision.

Once retractor 210 has been lowered to a desired relationship relative to the spine 100 or other body tissue, a user may slide a connector 260 over each guide 240, 250 such that connector 260 is lowered to a connecting position. In the embodiment shown in FIGS. 10A-11B, connector 260 connects a guide 240, 250 to frame 220 by associating an attachment portion 262 with an indentation on the frame 222, and concurrently releasably fixing the connector 260 with a guide 240, 250. Other embodiments of a connector 260 (other than the embodiment shown herein, and in detail in FIG. 15, infra) are expressly contemplated, and will be appreciated by those skilled in the art.

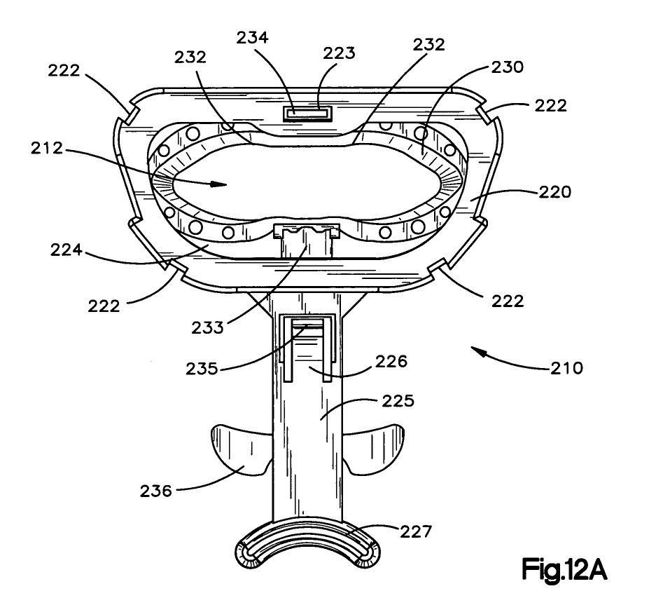

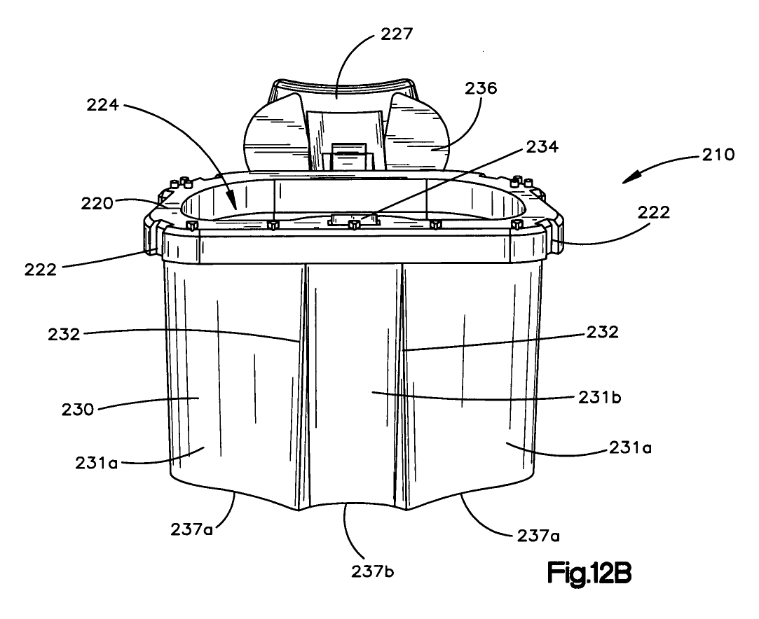

After connectors 260 are in place, the blade portion 230 of retractor 210 may be opened to enlarge opening 212 to a desired amount. Blade portion 230 may be opened similar to the methods described above. Preferably, blade portion 230 is associated with frame 220 such that blade portion 230 is configured to maintain an open position once it is urged into that open position. This may be achieved by the use of a locking/opening mechanism 40 as described above, or a variation of such a mechanism. The force required to open blade portion 230 may be substantially aligned with the direction of the displacement of at least a portion of the blade portion 230. An example of this may be seen by comparing FIGS. 11A-12A, as a portion of blade portion 230 has been displaced in the direction of force applied to gripping portion 236, as seen in FIGS. 11B-12A.

FIG. 12A shows a top view of the retractor 210 with blade portion 230 in an open position. As seen in illustrated embodiment, frame 220 may have plurality of indentations 222 for associating with a connector 260. Any suitable number of indentations 222 are contemplated, and the placement, shape, and size of the indentations 222 may be varied. Frame 220 may also have a slot 223 for receiving a tab portion 234 of blade portion 230. The interaction of slot 223 and tab portion 234 may serve to releasably secure frame 220 to blade portion 230. Frame 220 and blade portion 230 may also be secured by way of a slot (not shown) within blade portion 230 and a hook element (not shown) projected from frame 220. Various other securing and fixing relationships between frame 220 and blade portion 230 are contemplated to ensure that frame 220 and blade portion 230 are sufficiently secured during use. Frame 220 may also have an opening 224, which may be larger than the maximum size of opening 212. Frame 220 may also have a handle portion 225, which may have a locking/opening assembly 226 and a flange 227. Locking/opening assembly 226 may be associated with a contoured portion 235 of a blade portion 230, and may function substantially similarly to locking/opening mechanism 40, described above. Flange 227 may be useful in gripping the handle portion 225 of the frame 220.

Blade portion 230 may have at least one blade, and may have at least one hinge 232 disposed between blades. Hinge 232 may have any or all of the characteristics of a “living hinge,” as described above. Blade portion 230 may also have a projecting portion 233, which may have a contoured portion 235 and a gripping portion 236. As stated above, contoured portion 235 may interact with locking/opening assembly 226 of frame 220 to control the opening of blade portion 230, and also allow the blade portion 230 to be releasably locked in an open position. In the embodiment shown in FIG. 12A, this is achieved by providing a resilient tab in locking/opening assembly 226 and a series of parallel ridges on contoured portion 235, wherein as gripping portion 236 is pulled toward flange 227, resilient tab may engage subsequent rows of ridges on contoured portion 235. Moreover, ridges may be designed such that the resilient tab may not “back-our” of engagement with a subsequent ridge and return to a previous ridge, so that a unidirectional relative movement between the locking/opening assembly 226 and contoured portion 235 is maintained. The result of this arrangement may therefore be that as blade portion 230 is opened, it's opening 212 may not thereafter be reduced in size unless a user intervenes.

FIG. 12B shows a front view of the retractor of FIG. 12A, showing blade portion 230 in more detail. Blade portion 230 may have major blades 231a and minor blades 231b separated by hinges 232. Major blades 231a may be larger in size than minor blades 231b. Blade portion 230 may have any suitable number of major blades 231a, minor blades 231b, and/or hinges 232, the number of which may be varied at least in part on the desired shape and size of opening 212, and the amount of flexibility desired by the blade portion 230. Major and minor blades 231a, 231b may also each have curved lower edges 237a, 237b, which may assist the insertion process into an incision.

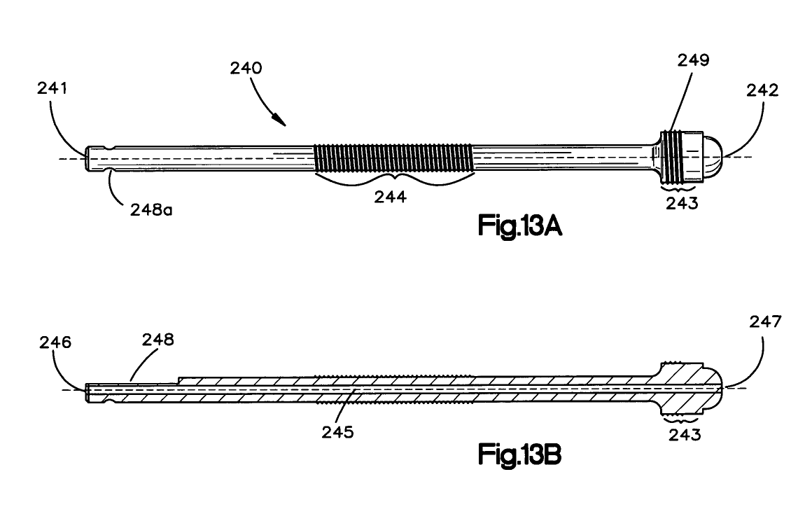

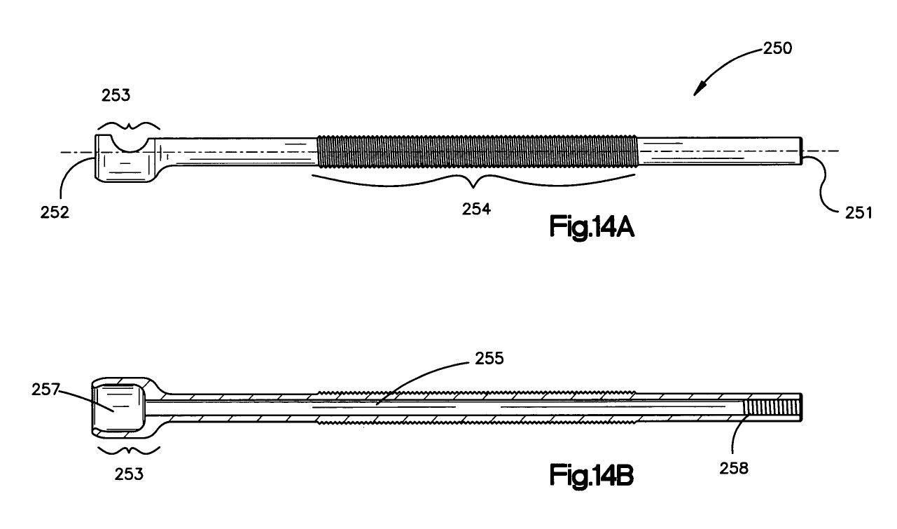

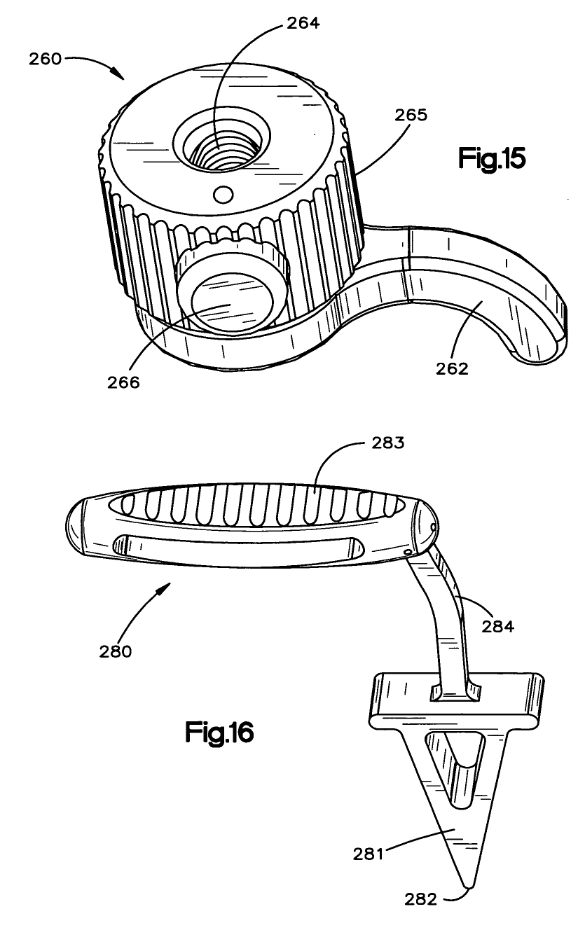

FIGS. 13A-14B show embodiments of guides 240, 250 for use with retractor 210. As seen in the side view of FIG. 13A, and cross-sectional view of FIG. 13B, guide 240 may have an attachment end 242, a free end 241, and a shaft extending therebetween having a threaded portion 244. Guide 240 may also have a bore 245 extending between a leading opening 247 and a trailing opening 246. Bore 245 may be beneficial for accepting a guide wire (not shown). Guide 240 may further have a bulbous attachment portion 243 located adjacent to the attachment end 242. Attachment portion 243 may directly or indirectly be associated with a bone screw 270, and may do so by way of a threaded portion 249. Guide 240 may also have a flattened portion 248 and an annular groove 248a, which may allow for releasably lockable insertion into an insertion instrument (not shown).

FIGS. 14A-14B show another embodiment of a guide 250. As seen in the side view of FIG. 14A, and cross-sectional view of FIG. 14B, guide 250 may have an attachment end 252, a trailing end 251, and a shaft extending therebetween having a threaded portion 254. Guide 250 may also have a bore 245 extending between a leading opening 257, and terminating at end 258. Guide 250 may further have an attachment portion 253 located adjacent attachment end 252. Attachment portion 253 may be an open-faced chamber for direct or indirect association with a bone screw 270. Though described independently, guides 240, 250 may have any of the characteristics of the other. Further modifications and combinations will be appreciated by those skilled in the art.

An embodiment of a connector 260 is shown in FIG. 15. Connector 260 may have an attachment portion 262 for association with a frame 220. Connector 260 may also have a central bore 264 for receiving a guide 240, 250. Central bore 264 may be provided by coextensive openings in attachment portion 262 and nut 265. Nut 265 may house a compression nut 266.

To adjust the clearance within central bore 264, compression nut 266 may be depressed and released. Specifically, when compression nut 266 is depressed by a user relative to nut 265, central bore 264 may be substantially free from obstruction, such that a guide 240, 250 may be passed through bore 264. Upon release of compression nut 266, it may be urged back it its original position by a compression spring (not shown) housed within nut 265, and bear against the guide 240, 250 within bore 264 such that guide 240, 250 is fixed within bore 264. Compression nut 266 may also have a threaded bore (not shown) for threaded engagement with a threaded portion 244, 254 of guides 240, 250. Other methods and arrangements of releasable securement between guides 240, 250 and connector 260 is expressly contemplated, as will be appreciated by those skilled in the art.

An embodiment of a wedge-spreader 280 is shown in FIG. 16. Wedge-spreader may have any or all of the same design characteristics and advantages of expander 180 described and shown in FIG. 7, above. Wedge-spreader 280 may have a wedge 281 having a leading edge 282, a handle 283, and a connecting portion 284 therebetween. Wedge-spreader 280 may be beneficial in assisting with the opening of blade portion 230.

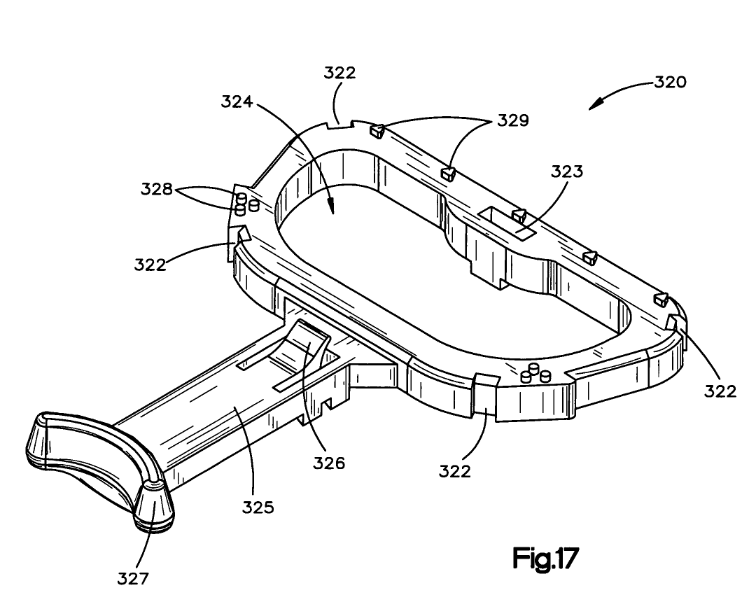

Another embodiment of frame 320 is shown in FIG. 17. Similar to frame 220 discussed above, frame 320 may have indentations 322 for association with a connector 260, a slot 323 for receiving a tab portion 235 of a blade portion, and an opening 324. Frame 320 may further have a handle portion 325 having a locking/opening assembly 326 having a resilient tab, and a flange 327. Frame 320 may further have raised projections 328 and 329 as well disposed along the outer perimeter of the frame. Projections 328, 329 may be beneficial in providing suitable locations along the frame 320 for engagement with a clamp (not shown) that may be utilized to affix the frame 320 to an external structure. Projections 328, 329 may also be useful for affixing a light device, suction device, nerve root effector, or other instrument to the frame 320.

Though several embodiments of the retractors discussed above are described for use with at least one guide, it is expressly contemplated that all retractors described herein may be used without the use of at least one guide, or any other attachment mechanism. Thus, a surgeon may find it preferable to utilize the retractor of FIGS. 1-2, 12A-12B without the use of any mechanism or components to attach the retractor to the body before or during operation of the retractor. In using the retractor in this way, a surgeon may make an incision in a desired location, and then insert the retractor directly into the incision. This procedure may be preferable when the desired retraction area is not overly deep, or does not involve undue force to retract body tissue in such retraction area. This procedure may also be preferable when is difficult or impractical to attach guides near or in the retraction area prior to or during insertion of the retractor.

Thus, specific embodiments and applications of novel retractors have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed:

1. A method of accessing a desired body site adjacent first and second bone surfaces comprising the steps of:
(a) inserting a first bone fastener into a first bone surface;
(b) inserting a second bone fastener into a second bone surface;
(c) associating a first guide with the first bone fastener;
(d) associating a second guide with the second bone fastener;
(e) lowering a device having a frame and a central opening over the first and second guides, such that the first and second guides extend through the central opening;
(f) associating the first and second guides with the frame; and
(g) manipulating the device to access the desired body site.

2. The method of claim 1, wherein the first guide is pivotably associated with the first bone fastener.

3. The method of claim 1, wherein the device further comprises a blade portion, and wherein the first and second guides extend through the blade portion in step (e).

4. The method of claim 1, wherein the first and second guides are associated with the frame at different locations.

5. The method of claim 1, wherein the first guide is associated with the frame with a first connector.

6. The method of claim 5, wherein the first connector comprises a bore, and wherein the first guide extends through the bore.

7. A method of accessing a desired portion of the spine comprising the steps of:
(a) inserting a first bone screw into a first vertebrae;
(b) inserting a second bone screw into a second vertebrae;
(c) associating a first guide with the first bone screw;
(d) associating a second guide with the second bone screw;
(e) lowering a retractor over the guides to a desired depth;
wherein the retractor comprises a frame, and a blade portion having a lower edge;
wherein the lower edge is adjacent the desired portion of spine when the retractor is at the desired depth;
(f) associating the first and second guides with the frame; and
(g) manipulating the blade portion to access the desired portion of the spine.

8. The method of claim 7, wherein the lower edge of the blade portion is substantially curved.

9. The method of claim 7, wherein the blade portion further comprises a first portion and a second portion, wherein the first portion is opposite the second portion, and wherein the first portion is urged away from the second portion in step (g).

10. The method of claim 7, wherein the blade portion is removably attached to the frame.

11. The method of claim 7, wherein an access opening is created in step (g).

12. The method of claim 11, further comprising the step of performing a surgical activity at the desired portion of the spine.

13. The method of claim 11, further comprising the step of inserting an instrument through the access opening.

14. The method of claim 11, wherein step (a) further comprises creating an access port with a cannula prior to inserting the first bone screw into the first vertebrae.

15. A method of accessing a desired body site comprising the steps of:
(a) providing a device configured to be manipulated from a closed position to an open position, and having a central opening;
(b) associating at least a first guide near the desired body site;
(c) lowering the device over the first guide such that the first guide passes through the central opening;
wherein the device is in a closed position;
(d) associating the first guide with the device; and
(e) manipulating the device to an open position.

16. The method of claim 15, wherein the device has a substantially linear leading edge when in a closed position.

17. The method of claim 15, wherein the device comprises a first opening in the closed position, wherein the device comprises a second opening in the open position, and wherein the second opening is larger than the first opening.

18. The method of claim 15, further comprising the step of making an incision in a patient prior to step (c).

19. A method of accessing a desired body site comprising the steps of:
(a) providing a device configured to be manipulated from a closed position to an open position, wherein the device comprises a blade portion and a frame, wherein the blade portion comprises a substantially continuous insertion portion, and wherein the insertion portion forms a substantially linear leading edge when the device is in a closed position;
(b) making an incision in a patient;
(c) inserting at least a portion of the insertion portion into the patient through the incision when the device is in a closed position; and
(d) manipulating the device to an open position by applying a force, such that a portion of the insertion portion is urged in a direction substantially aligned with the direction of the force.

20. The method of claim 19, wherein the insertion portion comprises a living hinge.
